(19) United States
(12) Patent Application Publication
(10) Pub. No.: US 2008/0242876 A1
(43) Pub. Date: Oct. 2, 2008

(54) Process For the Manufacture of Substituted Propionic Acids

(75) Inventors: Peter McCormack, Merseyside (GB); Chen Weiping, Liverpool (GB); Mohammed Karim, Liverpool (GB)

Correspondence Address: KNOBBE MARTENS OLSON & BEAR LLP, 2040 MAIN STREET, FOURTEENTH FLOOR, IRVINE, CA, 92614, US

(73) Assignee: Phoenix Chemicals Ltd., Merseyside (GB)

(21) Appl. No.: 11/813,986

(22) PCT Filed: Jan. 13, 2006
(86) PCT No.: PCT/GB2006/000129

§ 371 Date: Jun. 10, 2008

(30) Foreign Application Priority Data
Jan. 14, 2005 (GB) 0500700.0

(51) Int. Cl. C07D 333/02; C07C 63/04

(52) U.S. Cl. 549/79; 562/493

(57) ABSTRACT

The invention concerns a process for the manufacture of substituted propionic acids comprising providing a substrate of formula (I): And subjecting the substrate to enantioselective hydrogenation under enantioselective hydrogenation conditions in the presence of an enantioselective hydrogenation catalyst comprising a catalyst ligand having a metallocene group with a chiral phosphorus or arsenic substituent to provide in enantiomeric excess a product of formula (II): or its enantiomer or if applicable its diastereomer.

This invention relates to an enantioselective process for synthesising certain substituted propionic acids.

WO-A-2005/068477 discloses certain classes of ligand useful in chiral catalysis, and WO-A-2005/068478 discloses processes for making these and other ligands.

WO-A-2002/02500 discloses a stereoselective synthesis of (R)-2-alkyl-3-phenylpropionic acids comprising the addition of suitably substituted propionic acid esters to suitably substituted benzaldehydes to form corresponding substituted hydroxy propionic acid esters, followed by the conversion of the hydroxyl group to a leaving group, elimination of the leaving group, hydrolysis and then hydrogenation of the resulting intermediates.

Sturm et al disclose in Adv. Synth. Catal. 2003, 345, 160-164 a series of diphosphines of the Walphos ligand family and the use thereof in enantioselective hydrogenation.

WO-A-2005/030764 and Organic Letters 2005, vol 7, pp 1947 disclose processes for the preparation of chiral propionic acid derivatives.

According to the present invention, there is provided a process for the manufacture of substituted propionic acids comprising providing a substrate of formula (I):

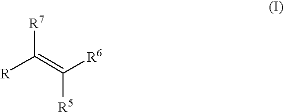

wherein:
R is selected from hydrogen, substituted and unsubstituted branched and straight-chain alkyl, alkoxy, alkylamino, substituted and unsubstituted cycloalkyl, substituted and unsubstituted cycloalkylamino, substituted and unsubstituted carbocyclic aryl, substituted and unsubstituted carbocylic aryloxy, substituted and unsubstituted heteroaryl, substituted and unsubstituted carbocylic arylamino and substituted and unsubstituted heteroarylamino, wherein the or each heteroatom is independently selected from sulphur, nitrogen and oxygen;
R⁵ is the same as or different from R and is selected from hydrogen, substituted and unsubstituted branched and straight-chain alkyl, alkoxy, alkylamino, N-acyl, substituted and unsubstituted cycloalkyl, substituted and unsubstituted cycloalkylamino, substituted and unsubstituted carbocyclic aryl, substituted and unsubstituted carbocylic aryloxy, substituted and unsubstituted heteroaryl, substituted and unsubstituted carbocylic arylamino and substituted and unsubstituted heteroarylamino, wherein the or each heteroatom is independently selected from sulphur, nitrogen and oxygen;
R⁶ is selected from:

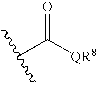

wherein:
Q is selected from O or N; and
R⁸ is selected from hydrogen, substituted and unsubstituted branched and straight-chain alkyl, amino, alkylamino, substituted and unsubstituted cycloalkyl, substituted and unsubstituted cycloalkylamino, substituted and unsubstituted carbocyclic aryl, substituted and, substituted and unsubstituted heteroaryl, substituted and unsubstituted carbocylic arylamino and substituted and unsubstituted heteroarylamino, wherein the or each heteroatom is independently selected from sulphur, nitrogen and oxygen;
R⁷ is the same as or different from R and/or R⁵ (except that if R and R⁷ are the same then R⁵ is not hydrogen) and is selected from hydrogen, substituted and unsubstituted branched and straight-chain alkyl, alkoxy, alkylamino, substituted and unsubstituted cycloalkyl, substituted and unsubstituted cycloalkylamino, substituted and unsubstituted carbocyclic aryl, substituted and unsubstituted carbocylic aryloxy, substituted and unsubstituted heteroaryl, substituted and unsubstituted carbocylic arylamino and substituted and unsubstituted heteroarylamino, wherein the or each heteroatom is independently selected from sulphur, nitrogen and oxygen; and
subjecting the substrate to enantioselective hydrogenation under enantioselective hydrogenation conditions in the presence of an enantioselective hydrogenation catalyst comprising a catalyst ligand having a metallocene group with a chiral phosphorus or arsenic substituent to provide in enantiomeric excess a product of formula (II):

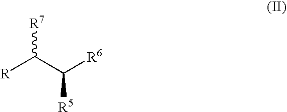

or its enantiomer or if applicable its diastereomer.

In one process according to the invention the substrate may be of formula (III):

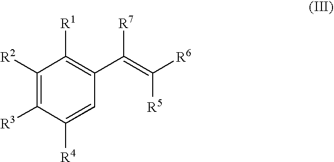

wherein R¹, R², R³ and R⁴ are the same or different and are independently selected from hydrogen, alkyl, haloalkyl, alkoxy, alkoxylated alkyl and alkoxylated alkoxy; the product of the process being of formula (IV):

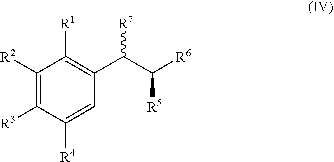

One particularly preferred process of the invention is for the manufacture of substituted arylpropionic acids, for example 2-substituted-3-arylpropionic acids, for example 2-alkyl-3-arylpropionic acids, such as 2-alkyl-3-phenylpropionic acids, particularly (R)-2-alkyl-3-phenylpropionic acids.

A preferred substrate for use in the process of the invention is a substrate of formula (V):

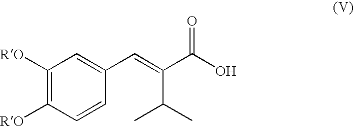

Wherein R′O is any suitable alkoxy or alkoxylated alkoxy group, and wherein each R′O may be the same or different.

Enantioselective hydrogenation if the formula (V) substrate in accordance with the invention yields a product of formula (VI):

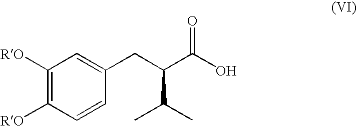

The process of the invention has been found suitable for enantioselectively hydrogenating the formula (I) substrates, and the other substrates referred to herein with good yields and reactions rates and, importantly, with high enantiomeric excesses of the desired enantiomer. Certain characteristics of the catalyst are considered to be important in achieving good ee's. Thus, in some cases it is preferable that the metallocene group of the catalyst ligand comprise ortho to the chiral phosphorus or arsenic substituent a second chiral substituent group. It may also be desirable in some cases that the chiral phosphorus or arsenic substituent on the metallocene group be further connected via a linking moiety to a second chiral phosphorus or arsenic substituent on a second metallocene group in the catalyst ligand. In this case it is also preferred that the chiral configuration of the chiral phosphorus or arsenic substituent is the same as the chiral configuration of the second chiral phosphorus or arsenic substituent. Still other catalyst characteristics may also be important and in some cases it has been found desirable that the catalyst ligand exhibit C₂ symmetry. Yet a further desirable characteristic of the catalyst ligand in some cases is that it be basic, for example as a result of the ability to donate one or more loan pairs from one or more nitrogen-containing substituents.

One preferred enantioselective hydrogenation catalyst ligand has the formula (VII):

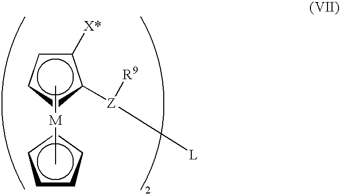

wherein:
M is a metal;

### Z is P or As;

L is a suitable linker;
R⁹ is selected from substituted and unsubstituted, branched- and straight-chain alkyl, alkoxy, alkylamino, substituted and unsubstituted cycloalkyl, substituted and unsubstituted cycloalkoxy, substituted and unsubstituted cycloalkylamino, substituted and unsubstituted carbocyclic aryl, substituted and unsubstituted carbocyclic aryloxy, substituted and unsubstituted heteroaryl, substituted and unsubstituted heteroaryloxy, substituted and unsubstituted carbocyclic arylamino and substituted and unsubstituted heteroarylamino, wherein the or each heteroatom is independently selected from sulphur, nitrogen, and oxygen;
X* is selected from:

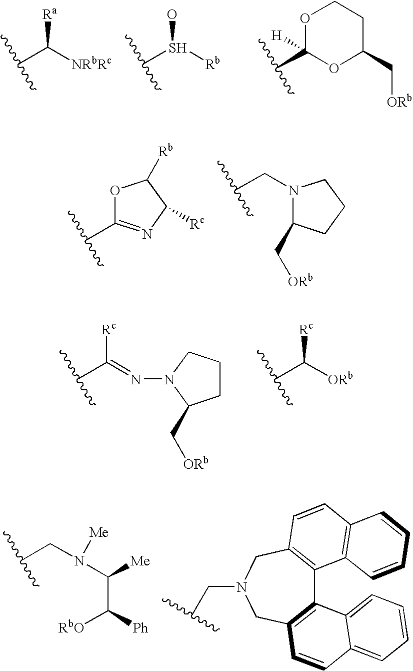

wherein R^{a}, R^{b} and R^{c} are independently selected from substituted and unsubstituted, branched- and straight-chain alkyl, substituted and unsubstituted cycloalkyl, substituted and unsubstituted carbocyclic aryl, and substituted and unsubstituted heteroaryl wherein the or each heteroatom is independently selected from sulphur, nitrogen, and oxygen.

In the first of the structures defining X*, R^{b} and R^{c} may form, together with the nitrogen to which they are attached, an optionally substituted hetero-ring, such as morpholine, pyrollidine, piperidine, and derivatives thereof.

L preferably comprises a difunctional moiety having the capability at each functionality to bind to phosphorus or arsenic, as the case may be. Generally the linker (L) will be derived from a difunctional compound, in particular a compound having at least two functional groups capable of binding to phosphorus or arsenic, as the case may be. The difunctional compound may conveniently comprise a compound which can be di-lithiated or reacted to form a di-Grignard reagent, or otherwise treated, to form a dianionic reactive species which can then be combined directly with phosphorus or arsenic, in a diastereoselective manner to form a chiral phosphorus or arsenic as the case may be. In this case, a first anionic component of the dianionic reactive species may combine with a phosphorus (or arsenic) substituent in a first ligand precursor of the ligand according to the invention, and a second anionic component of the dianionic reactive species may combine again in a diastereoselective manner with a phosphorus (or arsenic) substituent in a second ligand precursor of the ligand again to form a chiral phosphorus (or arsenic) centre according to the invention (the first and second ligand precursors being the same as each other) to connect the first and second ligand precursors together via the linker. Usually a leaving group such as a halide will be provided on the phosphorus (or arsenic) substituents of the first and second ligand precursors, which leaving group departs on combination of the anionic component with the phosphorus (or arsenic) substituent. The following scheme is illustrative of this process:

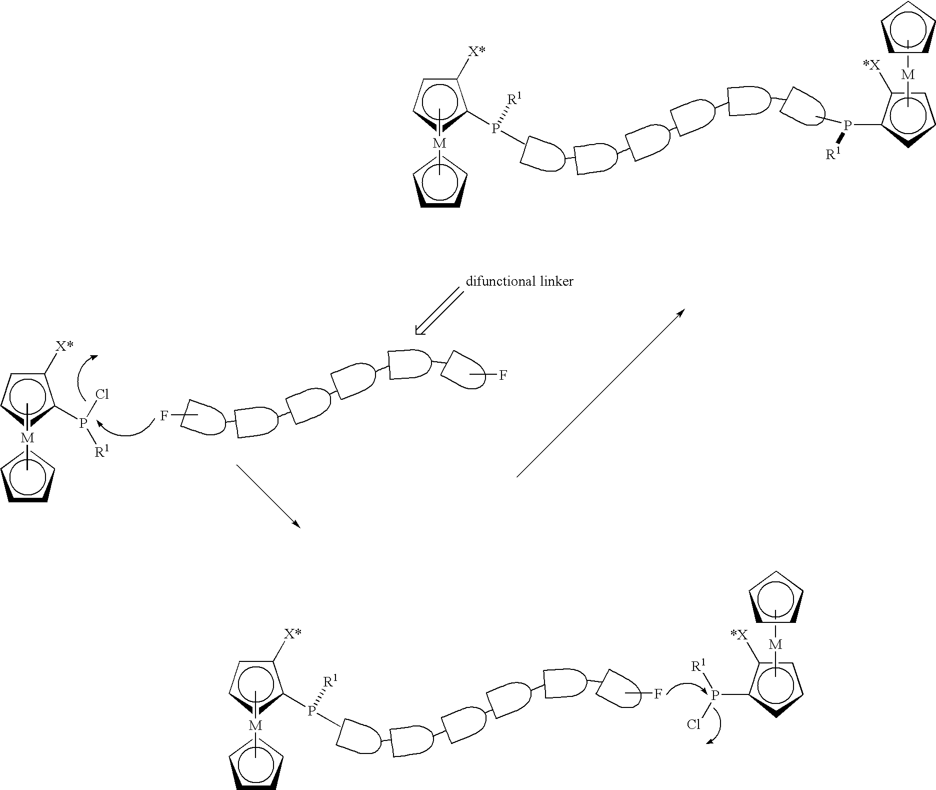

For example, L may be selected from ferrocene and other metallocenes, diphenyl ethers, xanthenes, 2,3-benzothiophene, 1,2-benzene, succinimides, cyclic anhydrides and many others. Conveniently, although not necessarily such dianionic linkers may be made from a corresponding di-halo precursor, eg:

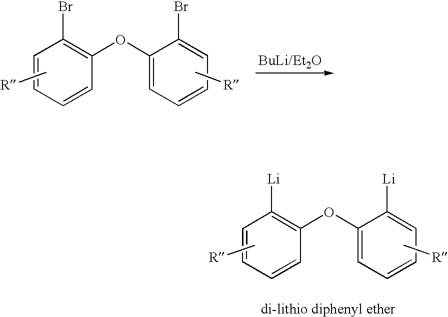

where R″ represents any suitable number of suitable substituent groups. Certain suitable dianionic linkers (wherein again R″ is simply any suitable number of any suitable substituent(s)) may be represented as follows:

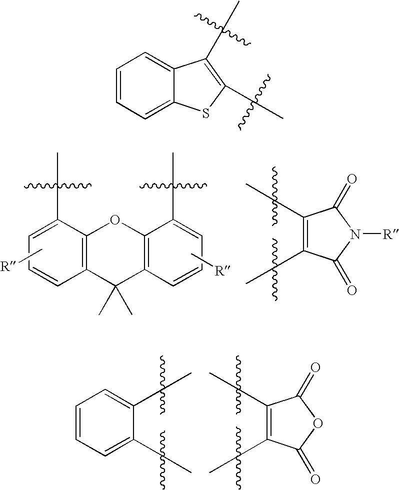

However, ferrocene is a preferred linker in accordance with the invention.

Preferably M is Fe, although Ru may be another preferred M in some cases.

Preferred R⁹ include phenyl, methyl, cyclohexyl and t-butyl groups.

Preferred R^{b} and R^{c} include, independently, methyl, ethyl, isopropyl and t-butyl groups. Also, R^{b} and R^{c} may form, together with the nitrogen to which they are attached, an optionally substituted hetero-ring such as morpholine, pyrollidine, piperidine, and derivatives thereof.

With very many known ligands for asymmetric hydrogenation of substrates of formula (V) enantoselectivities of 80% are achieved (Adv. Synth. Catal. 2003, 345, 160). In the same paper Sturm and in WO 02/02500 A1 Herold disclose that certain ligands of the Walphos family can furnish enantioselectivites of 95% for substrates of formula (V). It has been surprisingly found that certain ligands described here of general formula (VII) are especially useful for the enantioselective hydrogenation of substrates of formula (V) and can furnish with industrially useful reaction rates enantioselectivites of up to 99% or more. This improvement can offer significant cost savings during industrial manufacture of compounds of formula (VI) or their enantiomers.

Similarly certain of the ligands described here are also suitable as catalysts in combination with an appropriate metal for the enantioselective hydrogenation of substrates (in which R′″ is any suitable substituents such as substituted and unsubstituted, branched- and straight-chain alkyl, substituted and unsubstituted cycloalkyl, substituted and unsubstituted carbocyclic aryl, and substituted and unsubstituted heteroaryl, wherein the or each heteroatom is independently selected from sulphur, nitrogen, and oxygen, for example) of formula (VIII).

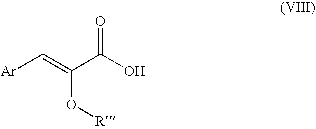

Thus compounds such as formula (IX) are also accessible in high enantioselectivity using the ligands and processes described here.

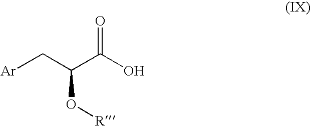

Certain ligands useful in the process of the invention are derived from Ugi's amine and one preferred ligand for use in accordance with the process of the invention (wherein the dianionic linker is ferrocene) may be represented as:

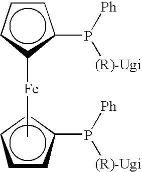

The same preferred ligand, with the Ugi amine groups fully represented may be shown as:

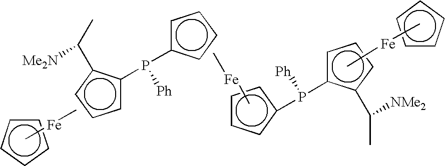

The ligand above has three chiral elements; carbon centred chirality, phosphorus centred chirality and planar chirality with two examples of each type present in the ligand. Due to its symmetry (C₂ symmetric) these elements are in two identical groups 2(S_{P},R_{C},S_{Fe}) where the labels R or S have their usual meaning and where S_{P} refers to phosphorus centred, R_{C} carbon centred and S_{Fe} planar chirality.

The invention also relates to the use of enantiomers and diastereomers of the ligands described above in the process of the invention.

Ligands used in the process of the invention may also be represented as: follows:

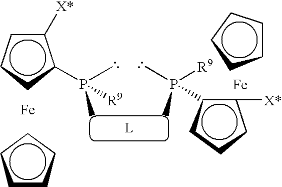

Wherein M, L, R⁹ and X* are as previously defined.

Also provided in. accordance with the invention is the use in the process of the invention of a transition metal complex comprising at least one transition metal coordinated to the aforementioned ligand. The metal is preferably a Group VIb or a Group VIII metal, especially rhodium, ruthenium, iridium, palladium, platinum and nickel.

Synthesis of ferrocene-based phosphorus chiral phosphines may be effected in accordance with the following scheme:

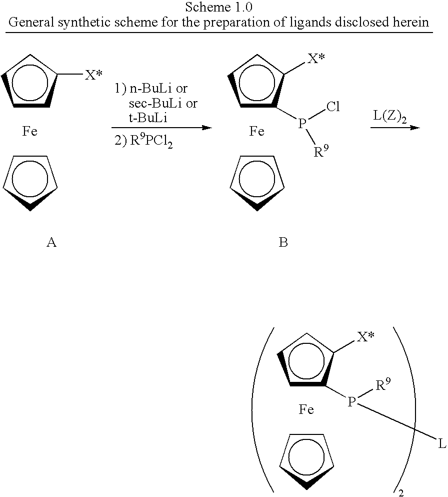

wherein L is a linker derived from an organolithium species or Grignard reagent L(Z)₂ and wherein X* and R⁹ are as previously defined. The organodilithium or di-Grignard reagent (the linker L(Z)₂ in the above scheme) adds to the chlorophosphine intermediate B to generate a phosphorus chiral centre with very good diastereoselectivity as is shown in WO2005/068478 A1. Other reactions used in the synthesis of these ligands are known or are analogous to known reactions. The same synthetic scheme is generally applicable to other chiral metallocene-based ligands for use in accordance with the invention.

The metal complexes used as catalysts can be prepared and isolated separately and then added to the reaction or they can be prepared in-situ before the reaction (not isolated) and then mixed with the material to be hydrogenated. It has been unexpectedly found that with the ligands described here there is no need to pre-form (either in-situ or separately with isolation) the catalyst by mixing a solution of the ligand and metal source when carrying out enantioselective hydrogenations of the acid substrates described here. Thus conveniently, all the solid materials (ligand, metal source and substrate) required for reaction can be placed in the vessel, the solvent is transferred, the vessel placed under the required temperature and pressure and the reaction commenced. In this way it is convenient to add extra ligand, other ligands and/or other additives to the reaction. Additives such as protic acids and quaternary ammonium halides can be used as co-catalysts.

The enantioselective hydrogenation reaction can be carried out at any suitable temperature, for example temperatures of from about 0 to about 120° C., or from about 20 to about 80° C. for example.

The enantioselective hydrogenation reaction can be carried out at any suitable pressure, for example at hydrogen pressures of 5-200 bar.

The enantioselective hydrogenation reaction can be carried out using any suitable substrate to catalyst ration, for example with catalyst present in the reaction mixture in an amount of from about 0.0001 to about 10 mol % (with 100 mol % being the amount of material to be hydrogenated). The range 0.001 to 5 mol % is preferred with the range 0.01 to 1 mol % being particularly preferred.

The enantioselective hydrogenation reaction can be carried out with or without the use of a solvent. When a solvent is used it is preferably at least substantially inert with respect to the substrate and/or the catalyst. The solvent when present may comprise for example one or more of: alcohols (such as methanol, ethanol, propanol, butanol, ethylene glycol monomethyl ether, ethylene glycol monoethyl ether, diethylene glycol monomethyl ether), aliphatic, cycloaliphatic and aromatic hydrocarbons (pentane, hexane, petroleum ether, cyclohexane, methylcyclohexane, benzene, toluene, xylene), aliphatic halogenated hydrocarbons (dichloromethane, chloroform, diandtetrachloroethane), nitrites (acetonitrile, propionitrile, benzonitrile), ketones (acetone, methyl isobutyl ketone), carbonic esters and lactones (ethyl or methyl acetate,valerolactone), N-substituted lactams (N-methylpyrrolidone), carboxamides(dimethylamide, dimethylformamide), acyclic ureas (dimethylimidazoline), and sulfoxides and sulfones (dimethyl sulfoxide, dimethyl sulfone, tetramethylene sulfoxide, tetramethylene sulfone), water, and suitable mixtures of two or more thereof.

The invention will now be more particularly illustrated with reference to the following Examples. In these examples the synthesised substrates are in many cases themselves novel compounds. According to the present invention there is provided a novel compound having the structure indicated below in one or more of the following examples, and derivatives and close variants thereof.

### EXAMPLE 1

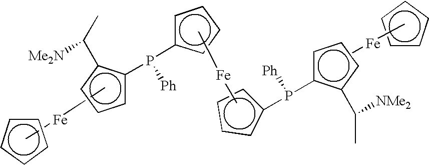

### 1,1′ bis-[(S_{P},R_{C},S_{Fe})(1-N,N-Dimethylamino)ethylferrocenyl)phenylphosphino]ferrocene L1

To a solution of (R)—N,N-dimethyl-1-ferrocenylethylamine [(R)-Ugi's amine] (3.09 g, 12 mmol) in Et₂O (20 ml) was added 1.5 M t-BuLi solution in pentane (8.0 ml, 12.0 mmol) at −78° C. After addition was completed, the mixture was warmed to room temperature, and stirred for 1.5 h at room temperature. The mixture was then cooled to −78° C. again, and dichlorophenylphosphine (1.63 ml, 12.0 mmol) was added in one portion. After stirring for 20 min at −78° C., the mixture was slowly warmed to room temperature, and stirred for 1.5 h at room temperature. The mixture was then cooled to −78° C. again, and a suspension of 1,1′ dilithioferrocene [prepared from 1,1′ dibromoferrocene (1.72 g, 5.0 mmol) and 1.5 M t-BuLi solution in pentane (14.0 ml, 21.0 mmol) in Et₂O (20 ml) at −78° C.] was added slowly via a cannula. The mixture was warmed to room temperature and allowed to stir for 12 h. The reaction was quenched by the addition of saturated NaHCO₃ solution (20 ml). The organic layer was separated and dried over MgSO₄ and the solvent removed under reduced pressure. The filtrate was concentrated. The residue was purified by chromatography (SiO₂, hexane-EtOAc-Et₃N=85:10:5) to afford an orange solid (3.88 g, 85%) as a mixture of 95% bis-(S_{P},R_{C},S_{Fe}) title compound L1 and 5% (R_{P},R_{C},S_{Fe}—S_{P},R_{C},S_{Fe}) meso compound. The meso compound can be removed by further careful purification using chromatography (SiO₂, hexane-EtOAc-Et₃N=85:10:5). Orange/yellow crystalline solid m.p. 190-192° C. [α]_{D}=−427° (c=0.005 (g/ml), toluene); ¹H NMR (CDCl₃, 400.13 MHz): δ 1.14 (d, 6H, J=6.7 Hz), 1.50 (s, 12H); 3.43 (m; 2H); 3.83 (m, 2H); 3.87 (m, 2H); 4.01 (s, 10H), 4.09 (t, 2H, J=2.4 Hz); 4.11 (m, 2H); 4.20 (m, 2H); 4.28 (m, 2H); 4.61 (m, 2H); 4.42 (d, 2H, J=5.3 Hz); 7.18 (m, 6H); 7.42 (m, 4H) ppm. ¹³C NMR (CDCl₃, 100.61 MHz): δ 38.28, 57.40 (d, J=5.6 Hz); 67.02, 69.04 (d, J=4.0 Hz); 69.16 (d, J=51.6 Hz); 69.66, 71.60 (d, J=4.8 Hz), 71.91 (d, J=7.2 Hz), 72.18 (d, J=5.6 Hz), 75.96 (d, J=35.7 Hz), 79.96 (d, J=6.4 Hz), 95.73 (d, J=19.1 Hz), 127.32 (d, J=7.9 Hz), 127.62, 133.12 (d, J=21.4 Hz), 139.73 (d, J=4.0 Hz). ³¹P NMR (CDCl₃, 162 MHz): δ-34.88 (s). Found: C, 65.53; H, 5.92; N, 3.01; Calculated for C₅₀H₅₄Fe₃N₂P₂; C, 65.81; H, 5.97; N, 3.07. HRMS (10 eV, ES+): Calcd for C₅₀H₅₅Fe₃N₂P₂ [M+H]⁺: 913.1889; Found: 913.1952.

The label S_{P} refers to S configuration at phosphorus, R_{C} refers to R configuration at carbon (or other auxiliary) and S_{Fe} refers to S configuration at the planar chiral element.

Note: To maintain consistency in all of this work when assigning configuration at phosphorus we have given the Ugi amine (1-N,N-dimethylamino)ethylferrocenyl) fragment a priority of 1, the incoming lithium or Grignard nucleophile (in the above example lithioferrocene) a priority of 2 and the remaining group a priority of 3. This method will not always be consistent with the rigorous approach. These assignations and the proposed phosphorus configurations have been checked using single crystal x-ray crystallography.

### EXAMPLE 2

### 2,2′ bis-[(S_{P},R_{C},S_{Fe})(1-N,N-Dimethylamino)ethylferrocenyl)phenylphosphino]-4-tolylether L2

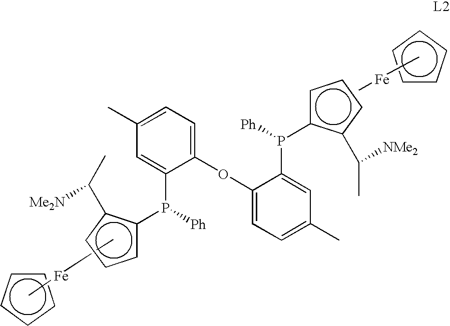

Using a similar procedure to that described above with the exception that a suspension of 2,2′ dilithio-4-tolylether [prepared by known procedures from 2,2′ dibromo-4-tolylether (1.78 g, 5.0 mmol) and 1.5 M t-BuLi solution in pentane (14.0 ml, 21.0 mmol) in Et₂O (20 ml) at −78° C.] was used as the linker reagent rather than 1,1′ dilithioferrocene.

Yellow crystalline solid [α]_{D}=−105° (c=0.005 (g/ml), toluene); ¹H NMR (CDCl₃, 400.13 MHz): δ 1.23 (d, 6H), 1.72 (s, 12H); 2.28 (s, 6H); 4.11 (s, 10H); 4.12 (m, 2H overlapping); 4.28 (m, 2H); 4.31 (m, 4H); 4.35 (m, 2H, overlapping); 7.00-7.30 (m, 14H) ppm. ³¹P NMR (CDCl₃, 162 MHz): δ-40.69 (br s) ppm.

### EXAMPLE 3

### 2,7-di-tert-butyl-4,5-bis-[(S_{P},R_{C},S_{Fe})(1-N,N-Dimethylamino)ethylferrocenyl)phenylphosphino]-9,9-dimethyl-9H-xanthene

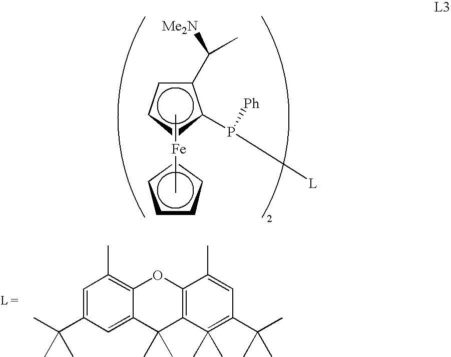

Using a similar procedure to that described above with the exception that a suspension of 2,7-di-tert-butyl-4,5-dilithio-9,9-dimethyl-9H-xanthene [prepared by known procedures from 2,7-di-tert-butyl-4,5-dibromo-9,9-dimethyl-9H-xanthene and 1.5 M t-BuLi solution in pentane in Et₂O at −78° C.] was used as the linker reagent rather than 1,1′ dilithioferrocene.

Orange/yellow crystalline solid; ¹H NMR (CDCl₃, 400.13 MHz): δ 1.12 (s, 18H); 1.13 (m, 6H overlapping); 1.78 (s, 6H); 1.98 (s, 12H); 3.99 (m, 2H); 4.15 (s, 10H overlapping); 4.32 (m, 2H); 4.41 (m, 4H); 7.00-7.40 (m, 14H) ppm. ³¹P NMR (CDCl₃, 162 MHz): δ-41.78 (br s) ppm. HRMS (10 eV, ES+): Calcd for C₆₃H₇₅Fe₂N₂OP₂ [M+H]⁺: 1049.4053; Found: 1049.4222

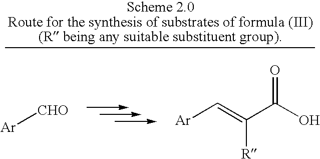

### EXAMPLE 4

### (E)-2-(4-methoxybenzylidine)-3-methylbutanoic acid

### Step 1

### Ethyl-2-hydroxy(4-methoxyphenyl)-methyl-3-methylbutanoate

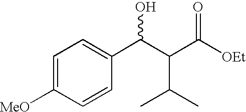

A solution of diisopropylamine (66 ml, 467 mmol) and anhydrous THF (394 ml) was cooled to (−30° C.). To this was added drop-wise n-butyl lithium (1.6 M, 292 ml) using syringe over a period of (20 min) and under stream of nitrogen. After addition of the n-BuLi, the reaction mixture was stirred at −30° C. for 10 min. Ethylisovalarate (55.8 ml, 428 mmol) in THF (250 ml) was added drop-wise over a period of (10 min). The reaction mixture was stirred for a further of 15 min then a solution of 4-methoxybenzaldehyde (34 g, 250 mmol) in THF (250 ml) was added over a period of 30 min at (maintaining temperature at −30° C.).The reaction mixture was stirred for 2 h at −30° C. and then saturated ammonium chloride (325 ml) was added drop-wise over a period of 30 min. The product was then extracted with EtOAc (200 ml), washed with brine and dried over sodium sulphate. Evaporation of the solvent under reduced pressure afforded a colourless oil 66.5 g (93%) which gave only one spot by TLC. m/z=[(ES) 289 (M+Na)⁺, 555 (2M+Na)⁺, calculated for C₁₅H₂₂O₄Na 289.1428, found 289.1426]. ¹H NMR (250 MHz, CDCl₃) δ 7.33-7.24 (2H, m, Ar), 6.92-6.84 (2H, m, Ar), 4.93 (1H, d), 3.93 (2H, q, CH₂CH₃), 3.89 (3H, s, OCH₃), 2.73 (1H, m), 2.44 (1H, m, CH), 2.40 (1H, m, OH), 1.19 (3H, t, CH₂CH₃), 1.17 (3H, d, CHCH₃), 1.15 (3H, d, CH₃), 1.13 (3H, d, CHCH₃).

### Step 2

### (E)-ethyl 2-(4-methoxybenzylidene)-3-methylbutanoate

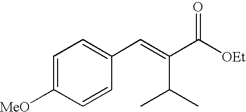

A solution of (31.56 g, 118 mol) of ethyl-2-hydroxy(4-methoxyphenyl)-methyl-3-methylbutanoate and dimethylaminopyridine (DMAP) (0.72 g, 5.9 mmol) in anhydrous THF (200 ml) were cooled to 0° C. using an ice bath. To this mixture was added acetic anhydride (12.3 ml, 12.5 mmol) drop-wise and then the reaction mixture was left stirring at 0° C. for 2 h. Potassium-t-butoxide (34.5 g, 350 mol) in 265 ml of THF was then added drop-wise using syringe. The reaction mixture was then stirred for two hours at 0° C. and overnight at room temperature. The mixture was then cooled to 0° C. and treated with water (150 ml). The mixture was extracted with TBME (100 ml), washed with brine and dried over sodium sulphate. Evaporation of the solvent under reduced pressure afforded a colourless light oil 18.52 g (63%).

### Step 3

### (E)-2-(4-methoxybenzylidine)-3-methylbutanoic acid

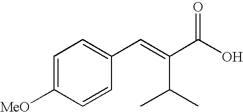

The oil from above (2-(4-methoxybenzylidine)-3-methoxyethylbutanoate) (16 g, 64.5 mmol) was dissolved in methanol (150 ml). To this was then added anhydrous lithium hydroxide (10 g, 417 mmol) at room temperature and the mixture was refluxed under a plug of nitrogen on oil bath for 12 h. The mixture was then cooled to 0-10° C. and quenched with water (100 ml). The basic solution was washed with EtOAc (3×50 ml) and then acidified with HCl (2 molar) and the precipitated product was extracted with EtOAc (3×50 ml), washed with brine and dried over sodium sulphate. Evaporation of solvent under reduced pressure afforded a solid residue this was then re-crystallised from EtOAc/hexane to afford 6.8 g (48%) of the title compound as white fine crystals, m.p. 137-138° C. ¹H NMR (250 MHz, CDCl₃) δ ppm: 11.50 (1H, br s, COOH), 7.71 (1H, s, CH═C), 7.34-7.38 (2H, m, Ar), 6.87-6.97 (2H, m, Ar), 3.81 (3H, s, OCH₃), 3.21 (1H, m, CH(CH₃)₂), 1.26 (6H, d, CH(CH₃)₂). M/z [(Cl) 221 (M+H)+45%, 238 (M+NH4) 100%]. Using a similar procedure to that described above the following compounds were prepared:

### EXAMPLE 5

### (E)-2-(4-Fluorobenzylidine)-3-methylbutanoic acid

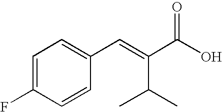

White crystalline solid. ¹H NMR (250 MHz, CDCl₃) δ ppm: 12.44 (1H, br s, COOH), 7.68 (1H, s, CH═C), 7.19-7.25 (2H, m, Ar), 6.99-719 (2H, m, Ar), 3.01-3.19 (1H, m, CH(CH₃)₂),1.33 (6H, d, CH(CH₃)₂).

### EXAMPLE 6

### (E)-2-((thiophen-2-yl)methylene)butanoic acid

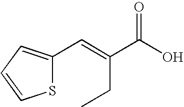

White crystalline solid M.p. 116-117° C.; ¹H NMR (250 MHz, CDCl₃) δ ppm: 12.46 (1H, br s, COOH), 7.92 (1H, s, CH═C), 7.47 (1H, m, Ar), 7.24 (1H, m, Ar), 7.08 (1H, m, Ar), 2.69 (2H, q, CH₂) and 1.25 (3H, s, CH₃) ppm.

### EXAMPLE 7

### (E)-3-methyl-2-((thiophen-2-yl)methylene)butanoic acid

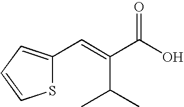

Beige crystalline solid. M.p. 116-117° C.; ¹H NMR (250 MHz, CDCl₃) δ ppm: 12.57 (1H, br s, COOH), 7.87 (1H, s, CH═C), 7.52 (1H, d, Ar), 7.26 (1H, d, Ar), 7.09 (1H, dd, Ar), 3.40-3.59 (1H, m, CH), 1.33 (6H, d, CH(CH₃)₂). M/z [(Cl) 196 (M)⁺ 10%, 197 (M+H)⁺ 30%, 214 (M+NH4)⁺ 100%].

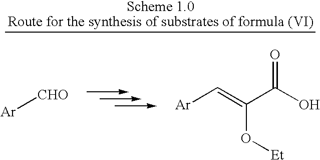

### EXAMPLE 8

### (Z)-2-Ethoxy-3-(thiophen-3-yl)acrylic acid

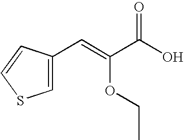

Ethyl chloroacetate (44.8 ml, 421 mmol) and anhydrous ethanol (30 ml) were cooled to 10-12° C. A solution of sodium ethoxide in ethanol (21% w/w, 165 ml) was added over 25 min at 12-16° C. under N₂. After addition was complete the reaction mixture was warmed to 25° C. and stirred for 1 h. The mixture was then cooled to 10° C. and solid NaOEt (33.3 g, 488 mmol) was then added portion-wise over 0.5 h at 10-14° C. Ethanol (20 ml) was then added followed by the addition of diethyl carbonate (31 ml, 256 mmol). The slurry was then cooled to 0-5° C. and then 3-thiophene carboxaldehyde (20.2 g, 179.5 mmol) was added over a period of 1 h. After addition was complete the mixture was stirred at 40° C. in an oil bath for 15 h. The slurry was then cooled to 10-15° C. and then water (40 ml) was added followed by the addition of aqueous NaOH (55 ml of a 10 M solution). The resulting slurry was then stirred at pH 14 for 3 h at 20° C. The mixture was then diluted with water (60 ml) and then placed under reduced pressure at 45° C. to remove most of the ethanol and some water. The resulting thick slurry was then cooled to 4° C. in an ice-bath and then treated with conc. HCl (115 ml) drop-wise. The resulting slurry was then stirred at room temperature for 1.5 h and then extracted with EtOAc (2×200 ml) and the organic layer washed with water, brine and then dried (sodium sulphate). Evaporation of the solvent under reduced pressure afforded a deep-brown residue. This was dissolved in 5 M NaOH (250 ml) and this solution was washed with EtOAc (100 ml). The basic aqueous was then cooled to 4° C. and acidified with conc. HCl (11M) to pH 4-6. The product was extracted with diethyl ether (3×200 ml), washed with brine, dried (sodium sulphate) and the solvent removed under reduced pressure. The residue was then filtered through a pad of silica (eluent hexane:EtOAc 90:10). The solvent was removed under reduced pressure and then the residue recrystallised from Et₂O/hexane to afford the title compound as yellow crystals (79%). M.p. 88-89° C. ¹H NMR (CDCl₃, 250 MHz) δ11.16 (1H, br s, COOH), 7.73-7.75 (1H, dd, j=0.5 Hz, Ar), 7.44-7.47 (1H, dd, J=1 Hz, Ar), 7.25-7.28 (1H, m, Ar), 7.18 (1H, s, CH═C), 3.96-4.05 (2H, q, J=7 Hz, CH₂CH₃), 1.35 (3H, t, J=7 Hz, CH₂CH₃)). Found: C, 54.64; H, 5.08; Calculated for C₉H₁₀SO₃ C, 54.54; H, 5.08. M/z [(Cl) 222 (M)⁺ 30%, 223 (M+H)⁺ 50%, 240 (M+NH₄)⁺ 100%; Found: 223.09705; required for C₁₂H15O₄ 223.09155]. M/z [(Cl) 198 (M)⁺ 22%, 199 (M+H)⁺ 50%, 216 (M+NH₄)⁺ 100%].

Using a similar procedure to that described above the following compounds were prepared:

### EXAMPLE 9

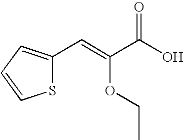

### (Z)-2-ethoxy-3-(thiophen-2-yl)acrylic acid

Pink crystalline solid (77%). M.p. 103-104° C. ¹H NMR (CDCl₃, 250 MHz) δ 12.15 (1H, br s, COOH), 7.48 (1H, s CH═C), 7.40 (1H, m, Ar), 7.29 ((1H, m, Ar), 7.08 (1H, m, Ar), 4.11 (2H, q, J=7 Hz, CH₂CH₃), 1.48 (3H, t, J=7 Hz, CH₂CH₃). Found: C, 54.82; H, 5.11, S, 16.00 Calculated for C₉H₁₀SO₃ C, 54.54; H, 5.08; S, 16.16]. M/z [(Cl) 222 (M)⁺ 30%, 223 (M+H)⁺ 50%, 240 (M+NH₄)⁺ 100%; Found: 223.09705; required for C₁₂H15O₄ 223.09155. M/z [(Cl) 198 (M)⁺ 22%, 199 (M+H)⁺ 50%, 216 (M+NH₄)⁺ 100%].

### EXAMPLE 10

### (Z)-3-(4-Cyanophenyl)-2-ethoxy acrylic acid

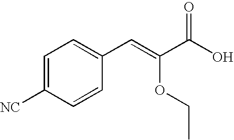

Following the procedure of (Vol. 8, No. 6, 2004, Organic Research & Development) with modification, this compound was synthesised as follows: Ethyl chloroacetate (44.5 ml, 421 mmol) and anhydrous ethanol (30 ml) were mixed and the solution cooled to 10-12° C. and treated slowly with NaOEt (21% w/w in EtOH, 165 ml, 421 mmol) over a period of 30 minutes. After the addition was complete, the reaction mixture was warmed to 25° C. and stirred for 1 h then cooled to 10° C. To this mixture was then added portion wise solid sodium ethoxide (33.5 g, 488 mmol) over a period of 0.5 h at 10-12° C. followed by addition ethanol (10 ml) and diethyl carbonate (31 ml, 256 mmol). The mixture was then cooled to 5-8° C. and then treated very slowly with 4-cyanobenzaldehyde (16.75 ml, 175 mmol) over a period of 1 h. After the addition of the reagent was complete, the reaction mixture was stirred on oil bath at 35° C. for 15 h. The slurry was then cooled to 15° C. and water (38 ml) was then added followed by the addition of sodium hydroxide (10 M, 55 ml, 55 mmol).The basic slurry at (pH 14) was stirred at 20° C. for 2.5 h. The mixture was diluted with water (120 ml) and most of the alcohol and some water was removed on rotary evaporator at 45° C. The resulting thick slurry was then diluted with water (105 ml) and cooled to 10-12° C. on ice bath. The slurry was then treated portion wise with dilute HCl (0.5 M, until pH 7) for a period of 1 h. The slightly acidic solution was then extracted with EtOAc (2×200 ml) washed with water, and then dried over sodium sulphate. After evaporation of the solvent the title compound was afforded as a solid and was re-crystallised from EtOAc-hexane to afford 21g (54%) as fine white crystals M.p. 171-172° C. ¹H NMR (CDCl₃, 250 MHz) δ 10.75 (1H, br s, COOH), 7.87 (2H, m, Ar), 7.67 (2H, m, Ar), 7.07 (1H, s, CH═C), 4.09-4.12 (2H, q, CH₂CH₃), 1.38 (3H, t, J=5 and 7.5 Hz, CH₂CH₃). Found: C, 66.28: H, 5.12; N, 6.42. Calculated for C₁₂H₁₁NO₃ C, 66.36; H, 5.09; NS, 6.45]. M/z [(Cl) 217 (M)⁺ 250%, 218 (M+H)⁺ 200%, 235 (M+NH₄)⁺ 100%.

### EXAMPLE 11

### (Z)-3-(3-(benzyloxy)-4-methoxyphenyl)-2-ethoxyacrylic acid

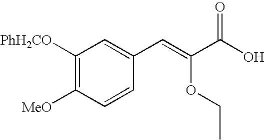

Pink crystalline solid. M.p. 147-148° C. ¹H NMR (CDCl₃, 250 MHz) δ 11.82 (1H, br s, COOH), 7.66 (1H, s CH═C), 7.24-7.57 (8H, m, Ar), 5.17 (2H, s, CH₂O), 3.83-3.99 (2H, q, CH₂CH₃), 3.94 (3H, s, OCH₃), 1.22-1.29 (3H, t, CH₂CH₃). Found: C, 69.40; H, 6.18, Calculated for C₁₉H₂₀O₅; C, 69.51; H, 6.15. M/z [(Cl) 328 (M)⁺ 20%, 329 (M+H)⁺ 45%, 346 (M+NH₄)⁺ 100%.

### EXAMPLE 12

### (Z)-3-(4-(benzyloxy)-3-methoxyphenyl)-2-ethoxyacrylic acid

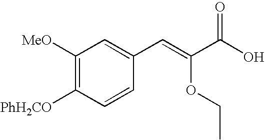

Pink crystalline solid. M.p. 148-149° C. ¹H NMR (CDCl₃, 250 MHz) δ 9.62 (1H, br s, COOH), 7.66 (1H, s, Ar), 7.11 (1H, s, (CH═C)), 7.10-7.45 (5H, m, Ar), 6.88 (2H, d, Ar), 4.17 (2H, q, CH₃CH₂), 3.94 (3H, s, OCH₃), 1.40 (3H, t, J=7 Hz, & J=5 Hz CH₂CH₃). Found: C, 69.27; H, 6.11; Calculated C₁₉H₂₀O₅; C, 69.51; H, 6.15. M/z [(Cl), 328 (M)⁺ 25%, 329 (M+H)⁺ 35%, 346 (M+NH₄)⁺ 100%.

### EXAMPLE 13

### (Z)-2-ethoxy-3-(3-methoxyphenyl)acrylic acid

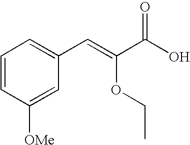

White crystalline solid. M.p. 99-100° C. ¹H NMR (CDCl₃, 250 MHz) δ 12.07 (1H, br s, COOH), 7.56 (1H, br s, Ar), 7.29 (2H, m, Ar), 7.15 (1H, s, CH═C), 6.92 (1H, m, Ar), 4.07 (2H, q, J=7.5 Hz, CH₂), 3.83 (3H, s, OCH₃), and 1.37 (3H, t, J=7 Hz). Found: C, 65.13; H, 6.37, Calculated for C₁₂H₁₄O₄; C, 64.86; H, 6.35. M/z [(Cl) 222 (M)⁺ 30%, 223 (M+H)⁺ 50%, 240 (M+NH₄)⁺ 100%; [Found: 223.09705; required for C₁₂H₁₅O₄; 223.09155].

### EXAMPLE 14

### General Hydrogenation Screening Method

Into a 45 ml autoclave was placed ligand (3.25×10⁻³ mM) and the vessel placed under vacuum/Ar cycles. The vessel was then flushed with Argon. A degassed solution of [(COD)₂Rh]BF₄ in MeOH (5 ml of a 0.64 mM solution) was then added by syringe/needle and a rubber bung placed over the vessel to maintain an inert atmosphere. This mixture was stirred for 10 min to give a clear yellow solution. A degassed solution of starting material in MeOH was then added by syringe/needle while carefully attempting to maintain an inert atmosphere. The autoclave was then connected to a Parr 3000 multi-vessel reactor system and then placed under Ar (5 bar) and vented while stirring, this process was repeated 3 times. After the final vent the mixture was placed under H₂ (50 bar) and again vented carefully. The mixture was then placed under H₂ (50 bar), sealed and heated to the desired temperature for the required time. After this time the reaction mixture was cooled and the vessel vented. An aliquot of 0.5-1.0 ml was then taken for analysis.

### EXAMPLE 15

### (S)-2-(3-(3-methoxypropoxy)-4-methoxybenzyl)-3-methylbutanoic acid

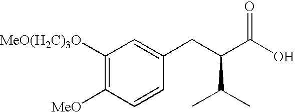

Into a 45 ml autoclave was placed 1,1′ bis-[(R_{P},S_{C},R_{Fe}) L1 (0.0063 g, 0.0069 mmol), [(COD)₂Rh]BF₄ (0.0025 g, 0.0061 mmol) and (E)-2-(3-(3-methoxypropoxy)-4-methoxybenzylidene)-3-methylbutanoic acid (2 g, 6.49 mmol). The vessel was then placed under vacuum/Ar cycles. The vessel was then flushed with Argon and a rubber bung placed over the vessel to maintain an inert atmosphere. Degassed MeOH (10 ml) was then added by cannula taking care to maintain an inert atmosphere in the vessel. The vessel was then sealed and stirring commenced. The vessel was then placed under Ar (5 bar) and vented, this process was repeated three times. The autoclave was then placed under H₂ (50 bar) and again vented carefully. The mixture was then placed under H₂ (50 bar), sealed and heated to 40° C. for 12 h. After this time the reaction mixture was cooled and the vessel vented. An aliquot of 0.5-1.0 ml was then taken for analysis. Conversion>98%, e.e>98.5% (major enantiomer second running peak).

¹H NMR (CDCl₃, 250.13 MHz): δ 1.01 (m, 6H), 1.95 (m, 1H); 2.05 (m, 2H); 2.45 (m, 1H); 2.78 (m, 2H); 3.35 (s, 3H), 3.55 (m, 2H); 3.83 (s, 3H); 4.10 (m, 2H); 6.65-6.80 (m, 3H).

### HPLC method for e.e. determination of 2-(3-(3-methoxypropoxy)-4-methoxybenzyl)-3-methylbutanoic acid

Chiralpak-AD column (250 mm×4.6 mm), 94% Hexane, 3% 2-methyl-2-propanol and 3% t-amyl alcohol, flow: 1 ml/min, 230 nm. S-acid 13.15 min (largest peak with bis-[(R_{P},S_{C},R_{Fe})]1), R-acid 14.01 min, starting material 42.73 min.

### HPLC method for e.e. determination of 2-(3-(3-methoxypropoxy)-4-methoxybenzyl)-3-methylbutanoic acid (methyl ester)-diazomethane derivatization

Into a 10 ml vial was placed a stirring bar and a 1 ml aliquot of the crude hydrogenation reaction mixture. With vigorous stirring trimethylsilyl diazomethane in hexane (2 M) was added drop-wise into the reaction mixture and the good yellow colour of the diazomethane solution disappeared along with good gas evolution. This drop-wise process was continued until the reaction mixture became a yellow colour and gas evolution ceased. Neat acetic acid (15-30 μl,—Caution too much acetic acid and excessive gas evolution occurs) was then added upon which the mixture became very pale yellow. Approximately ⅓ of this mixture was then filtered through a small pad of wetted silica in a Pasteur pipette washing with a little hexane/IPA (80:20). The resulting solution was then analysed using HPLC: Chiralpak-AD column (250 mm×4.6 mm), 95% Hexane, 5% i-Propyl alcohol, flow: 1 ml/min, 230 nm. Product enantiomers; 9-10 min, Starting material; 14-16 min. Note: the order of elution of the enantiomers is reversed relative to analysis on the non-derivatized acids.
1,1 bis-[(S_{P},R_{C},S_{Fe})] L1 yields (R)-2-(3-(3-methoxypropoxy)-4-methoxybenzyl)-3-methylbutanoic acid
1,1′ bis-[(R_{P},S_{C},R_{Fe})] L1 yields (S)-2-(3-(3-methoxypropoxy)-4-methoxybenzyl)-3-methylbutanoic acid

### EXAMPLE 16

TABLE 1.0
Results of enantioselective hydrogenations on (E)-2-(3-(3-
methoxypropoxy)-4-methoxybenzylidene)-3-methylbutanoic acid
with bis-[(S_{P},R_{C},S_{Fe})] L1 at 50 bar H₂ pressure.

| entry | s/c ratio | T (° C.) | Substrate [M] | Conversion (%) | e.e. (%) |
|---|---|---|---|---|---|
| 1 | 500:1 | 40 | 0.16 | >95 | 99.6¹ |
| 2 | 500:1 | 50 | 0.16 | >95 | 99.6² |
| 3 | 500:1 | 65 | 0.16 | >95 | 99.3² |
| 4 | 1000:1 | 40 | 0.55 | 72 | 98.5³ |
| 5 | 2000:1 | 40 | 0.55 | 72 | 98.3³ |

¹Reactions carried out in MeOH for 20 h
²Reactions carried out in MeOH for 5 h
³Reactions carried out in MeOH for 14 h

### EXAMPLE 17

TABLE 2.0
Results of enantioselective hydrogenations on (E)-2-(3-(3-
methoxypropoxy)-4-methoxybenzylidene)-3-methylbutanoic acid
with bis-[(S_{P},R_{C},S_{Fe})] L1 at 50 bar H₂ pressure.

| | | | | Solvent | |
|---|---|---|---|---|---|
| entry | s/c ratio | T (° C.) | Substrate [M] | MeOH:1-BuOH | e.e. (%) |
| 1 | 1000:1 | 40 | 0.65 | 8.75:1 | 98.7 |
| 2 | 1000:1 | 50 | 0.65 | 8.75:1 | 98.2 |
| 3 | 1000:1 | 65 | 0.65 | 8.75:1 | 96.6 |

### EXAMPLE 18

TABLE 3.0
Results of enantioselective hydrogenations on (E)-2-(3-(3-
methoxypropoxy)-4-methoxybenzylidene)-3-methylbutanoic acid
with bis-[(S_{P},R_{C},S_{Fe})] L1 at 50 bar H₂ pressure (using solid
addition method*)

| entry | Time (h) | T (° C.) | Substrate [M] | s/c ratio | e.e. (%) |
|---|---|---|---|---|---|
| 1 | 4 | 50 | 0.55 | 1000:1 | 98.6 |
| 2 | 4 | 60 | 0.55 | 2000:1 | 98.4 |
| 3 | 4 | 60 for 1 h then 50 | 0.55 | 1000:1 | 98.2 |

Note:
in all cases >98% conversion was observed
*All solids (substrate, ligand and metal source) placed in vessel then solvent added

### EXAMPLE 19

It has been found to be preferable for very high enantioselectivity that the meso impurity (R_{P},R_{C},S_{Fe}—S_{P},R_{C},S_{Fe})—L1 present in the ligand should be minimised.

TABLE 4.0
Results of enantioselective hydrogenations on (E)-2-(3-(3-
methoxypropoxy)-4-methoxybenzylidene)-3-methylbutanoic acid
with bis-[(S_{P},R_{C},S_{Fe})] L1 contaminated with meso impurity at 50 bar
H₂ pressure.

| | meso | | | | | |
|---|---|---|---|---|---|---|
| | present | T | Time | Solvent | Conversion | e.e. |
| entry | (%) | (° C.) | (h) | MeOH:1-BuOH | (%) | (%) |
| 1 | ~2 | 45 | 5 | 8.75:1 | 53 | 98.5 |
| 2 | ~2 | 55 | 5 | 8.75:1 | 92 | 98.2 |
| 3 | ~2 | 45 | 5 | 1:1.7 | 25 | 96.4 |
| 4 | 6-8 | 45 | 5 | 8.75:1 | 74 | 95.1 |
| 5 | 6-8 | 55 | 5 | 8.75:1 | >99 | 94.5 |
| 6 | 6-8 | 45 | 5 | 1:1.7 | 40 | 90.2 |

All reactions carried out at s/c ratio of 1000:1

### EXAMPLE 20

### Ligands containing flexible linker units have been found to be most preferable, for the enantioselective hydrogenation of the acid substrates described, eg

TABLE 5.0
Results of enantioselective hydrogenations on (E)-2-(3-(3-
methoxypropoxy)-4-methoxybenzylidene)-3-methylbutanoic acid
with ligands L1-L3 at 50 bar H₂ pressure in MeOH.

| | |
|---|---|
| | L2 |
| 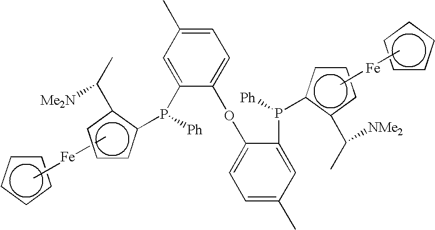 | |

| | | T | Time | | Conversion | e.e. |
|---|---|---|---|---|---|---|
| entry | Ligand | (° C.) | (h) | S/C ratio | (%) | (%) |
| 1 | L1 | 40 | 12 | 1000:1 | 83 | >99 |
| 2 | L2 | 40 | 12 | 1000:1 | 52 | 90.8 |

### EXAMPLE 21

### HPLC method for e.e. determination for (S)-2-ethoxy-3-(thiophen-2-yl)propanoic acid (as methyl ester)

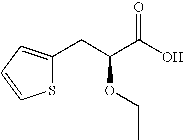

After derivatization:

Chiralpak-AD column (250 mm×4.6 mm), 95% Hexane, 2.5% 2-methyl-2-propanol and 2.5% t-amyl alcohol, flow: 1 ml/min, 236 nm. Enantiomers 5.44 and 5.81 min (largest peak with bis-[(S_{P},R_{C},S_{Fe})]1).

### EXAMPLE 22

### HPLC method for e.e. determination for (S)-3-(3-(benzyloxy)-4-methoxyphenyl)-2-ethoxypropanoic acid

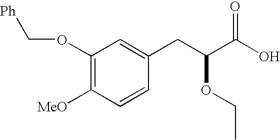

Chiralpak-AD column (250 mm×4.6 mm), 93% Hexane, 7% i-Propyl alcohol, flow: 1.2 ml/min, 235 nm. Enantiomers 11.71 min, 13.33 min (largest peak with bis-[(R_{P},S_{C},R_{Fe})]1), starting material 36.68 min.

### EXAMPLE 23

TABLE 6.0
Results of enantioselective hydrogenations on (Z)-[-(3-
Benzyloxy-4-methoxyphenyl)]-2-ethoxyacrylic acid with bis-
[(S_{P},R_{C},S_{Fe})] 1 at 48 bar H₂ pressure for 12 h.

| entry | s/c ratio | T (° C.) | Substrate [M] | e.e. (%) |
|---|---|---|---|---|
| 1 | 2000:1 | 50 | 0.40 | 96.2 |
| 2 | 2000:1 | 50 | 0.83 | 93.4 |
| 3 | 250:1 | 55 | 0.25 | 97.1 |
| 4 | 500:1 | 55 | 0.5 | 97.6 |
| 5 | 1000:1 | 55 | 1.0 | 94.9 |
| 6 | 1500:1 | 55 | 1.5 | 90.9 |
| 7 | 1000:1 | 80 | 1 | 81.2 |

All reactions carried out in MeOH
All reactions achieved >98% conversion

### EXAMPLE 24

### HPLC method for e.e. determination for (S)-2-ethoxy-3-(thiophen-3-yl)propanoic acid

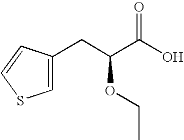

Chiralpak-AD column (250 mm×4.6 mm), 99% Hexane, 1% i-Propyl alcohol, flow: 0.7 ml/min, Integrated 235-239 nm. Enantiomers 9.71 min, 10.88 min (largest peak with bis-[(R_{P},S_{C},R_{Fe})]1), starting material 16.35 min.

### EXAMPLE 25

### HPLC method for e.e. determination for (S)-2-ethoxy-3-(3-methoxyphenyl)propanoic acid (as methyl ester)

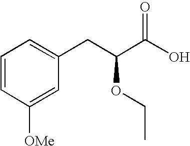

After derivatization:

Chiralpak-AD column (250 mm×4.6 mm), 95% Hexane, 2.5% 2-methyl-2-propanol and 2.5% t-amyl alcohol, flow: 1 ml/min, Integrated 280-290 nm.

Enantiomers 7.49 and 10.00 min (largest peak with bis-[(S_{P},R_{C},S_{Fe})]1).

### EXAMPLE 26

TABLE 7.0
Screening results of enantioselective hydrogenations on various
(Z)-substituted 3-aryl-2-ethoxyacrylic acid substrates with bis-
[(S_{P},R_{C},S_{Fe})] 1 at 50 bar H₂ pressure.

| entry | s/c ratio | T (° C.) | Substrate [M] | Substituted aryl | e.e. (%) |
|---|---|---|---|---|---|
| 1 | 500:1 | 40 | 0.41 | 3-OMe | 95.2 |
| 2 | 1000:1 | 40 | 0.82 | 3-OMe | 94.6 |
| 3 | 500:1 | 35 | 0.50 | 4-CN | 98.0 |
| 4 | 500:1 | 55 | 0.50 | 4-CN | 96.5 |
| 5 | 500:1 | 50 | 0.41 | 2-thienyl | 95.0 |
| 6 | 1000:1 | 55 | 0.41 | 3-thienyl | 96.5 |

All reactions carried out in MeOH

1. A process for the manufacture of substituted propionic acids comprising providing a substrate of formula (I): 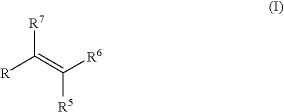
wherein:
R is selected from hydrogen, substituted and unsubstituted branched and straight-chain alkyl, alkoxy, alkylamino, substituted and unsubstituted cycloalkyl, substituted and unsubstituted cycloalkylamino, substituted and unsubstituted carbocyclic aryl, substituted and unsubstituted carbocylic aryloxy, substituted and unsubstituted heteroaryl, substituted and unsubstituted carbocylic arylamino and substituted and unsubstituted heteroarylamino, wherein the or each heteroatom is independently selected from sulphur, nitrogen and oxygen;
R⁵ is the same as or different from R and is selected from hydrogen, substituted and unsubstituted branched and straight-chain alkyl, alkoxy, alkylamino, N-acyl, substituted and unsubstituted cycloalkyl, substituted and unsubstituted cycloalkylamino, substituted and unsubstituted carbocyclic aryl, substituted and unsubstituted carbocylic aryloxy, substituted and unsubstituted heteroaryl, substituted and unsubstituted carbocylic arylamino and substituted and unsubstituted heteroarylamino, wherein the or each heteroatom is independently selected from sulphur, nitrogen and oxygen;
R⁶ is selected from: 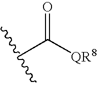
wherein:
Q is selected from O or N; and
R⁸ is selected from hydrogen, substituted and unsubstituted branched and straight-chain alkyl, amino, alkylamino, substituted and unsubstituted cycloalkyl, substituted and unsubstituted cycloalkylamino, substituted and unsubstituted carbocyclic aryl, substituted and unsubstituted heteroaryl, substituted and unsubstituted carbocylic arylamino and substituted and unsubstituted heteroarylamino, wherein the or each heteroatom is independently selected from sulphur, nitrogen and oxygen;
R⁷ is the same as or different from R and/or R⁵ (except that if R and R⁷ are the same then R⁵ is not hydrogen) and is selected from hydrogen, substituted and unsubstituted branched and straight-chain alkyl, alkoxy, alkylamino, substituted and unsubstituted cycloalkyl, substituted and unsubstituted cycloalkylamino, substituted and unsubstituted carbocyclic aryl, substituted and unsubstituted carbocylic aryloxy, substituted and unsubstituted heteroaryl substituted and unsubstituted carbocylic arylamino and substituted and unsubstituted heteroarylamino, wherein the or each heteroatom is independently selected from sulphur, nitrogen and oxygen; and
subjecting the substrate to enantioselective hydrogenation under enantioselective hydrogenation conditions in the presence of an enantioselective hydrogenation catalyst comprising a catalyst ligand having a metallocene group with a chiral phosphorus or arsenic substituent, the metallocene group comprising ortho to the chiral phosphorus or arsenic substituent a second chiral substituent group, to provide in enantiomeric excess a product of formula (II): 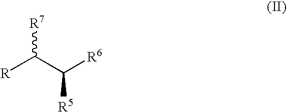
or its enantiomer or if applicable its diastereomer.

2. A process according to claim 1 wherein the substrate is of formula (III): 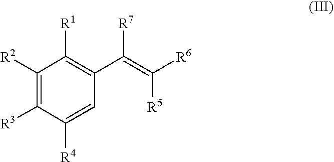
wherein R¹, R², R³ and R⁴ are the same or different and are independently selected from hydrogen, alkyl, haloalkyl, alkoxy, alkoxylated alkyl and alkoxylated alkoxy; the product of the process being of formula (IV):

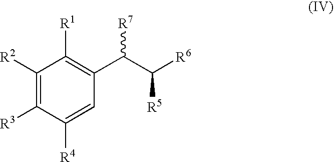

3. A process according to claim 2 wherein the substrate is a substrate of formula (V) 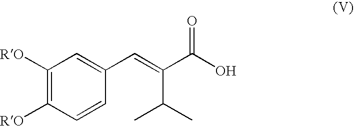
wherein R′O is any suitable alkoxy or alkoxylated alkoxy group, and wherein each R′O may be the same or different.

4. A process according to claim 3 wherein the product is a product of formula (VI):

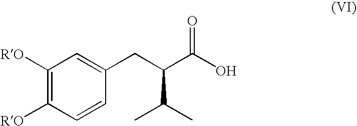

5. A process according to claim 1 wherein the chiral phosphorus or arsenic substituent on the metallocene group is further connected via a linking moiety to a second chiral phosphorus or arsenic substituent on a second metallocene group.

6. A process according to claim 5 wherein the configuration of the chiral phosphorus or arsenic substituent is the same as the configuration of the second chiral phosphorus or arsenic substituent.

7. A process according to claim 1 wherein the catalyst ligand exhibits C₂ symmetry.

8. A process according to claim 1 wherein the catalyst ligand is basic.

9. A process according to claim 1 wherein the catalyst ligand has the formula (VII): 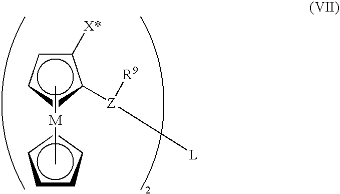
wherein:
M is a metal;
Z is P or As;
L is a suitable linker;
R⁹ is selected from substituted and unsubstituted, branched- and straight-chain alkyl, alkoxy, alkylamino, substituted and unsubstituted cycloalkyl, substituted and unsubstituted cycloalkoxy, substituted and unsubstituted cycloalkylamino, substituted and unsubstituted carbocyclic aryl, substituted and unsubstituted carbocyclic aryloxy, substituted and unsubstituted heteroaryl, substituted and unsubstituted heteroaryloxy, substituted and unsubstituted carbocyclic arylamino and substituted and unsubstituted heteroarylamino, wherein the or each heteroatom is independently selected from sulphur, nitrogen, and oxygen;
X* is selected from: 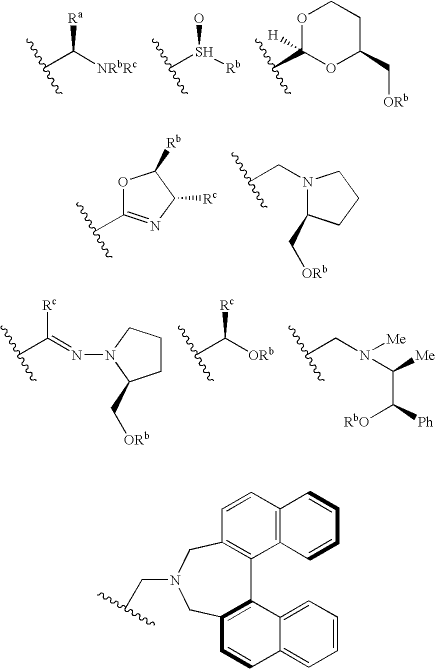
wherein R^{a}, R^{b} and R^{c} are independently selected from substituted and unsubstituted, branched- and straight-chain alkyl, substituted and unsubstituted cycloalkyl, substituted and unsubstituted carbocyclic aryl, and substituted and unsubstituted heteroaryl wherein the or each heteroatom is independently selected from sulphur, nitrogen, and oxygen.

10. A process according to claim 9 wherein R^{b} and R^{c} form, together with the nitrogen to which they are attached, an optionally substituted hetero-ring.

11. A process according to claim 9 wherein L the linker is derived from a dianionic reactive species.

12. A process according to claim 9 wherein L is selected from metallocenes, diphenyl ethers, xanthenes, 2,3-benzothiophenes, 1,2-benzenes, cyclic anhydrides and succinimides.

13. A process according to claim 12 wherein the linker comprises ferrocene.

14. A process according to claim 9 wherein the enantioselective hydrogenation catalyst comprises the enantiomer or diastereomer of a ligand having the formula (VII).

15. A process for the preparation of substituted propionic alcohols comprising preparing a substituted propionic acid by the process of claim 1, and then hydrogenating the acid.

16. A process for the preparation of substituted propionic halides comprising preparing a substituted propionic alcohol by the process of claim 15 and halogenating the alcohol.

17. A process for the preparation of substituted lactic acid comprising preparing by a process of claim 1 a substituted propionic acid of formula (II) wherein R⁵ is alkoxy and converting the alkoxy group to a hydroxy group.

18. A process according to claim 1 wherein the enantioselective hydrogenation catalyst comprises a transition metal coordinated to the catalyst ligand.

19. A process according to claim 18 wherein coordination between the transition metal and the catalyst ligand takes place in situ in the presence of the substrate.

20. A process according to claim 18 wherein the transition metal and the catalyst ligand are pre-coordinated before contact with the substrate.

21. A process according to claim 18 wherein the transition metal is a Group VIb or a Group VIII metal.

22. A process according to claim 21 wherein the transition metal is selected from rhodium, ruthenium, iridium, palladium, platinum or nickel.
